(19) United States
(12) Patent Application Publication
(10) Pub. No.: US 2025/0360496 A1
(43) Pub. Date: Nov. 27, 2025

(54) THIAZOLOTHIAZOLE CATALYSTS AND APPLICATIONS THEREOF

(71) Applicant: The University of North Carolina at Charlotte, Charlotte, NC (US)

(72) Inventors: Michael G. Walter, Charlotte, NC (US); Thomas Perrell, Charlotte, NC (US)

(21) Appl. No.: 19/217,805

(22) Filed: May 23, 2025

Related U.S. Application Data
(60) Provisional application No. 63/651,518, filed on May 24, 2024.

(51) Int. Cl. B01J 31/02; B01J 35/39; C07C 209/80; C07D 513/04

(52) U.S. Cl. CPC B01J 31/0271; B01J 35/39; C07C 209/80; C07D 513/04; B01J 2231/44

(57) ABSTRACT

Thiazolothiazole-based photocatalysts are described herein which, in some embodiments, exhibit advantageous redox potentials, including positive excited state redox potentials, for initiating coupling mechanisms involving one or more single electron transfers.

### RELATED APPLICATION DATA

The present application claims priority pursuant to 35 U.S.C. § 119(e) to U.S. Provisional Patent Application Ser. No. 63/651,518 filed May 24, 2024 which is incorporated herein by reference in its entirety.

### FIELD

The present invention relates to organic photocatalyst and, in particular, to thiazolothiazole photocatalysts providing advantageous redox properties.

### BACKGROUND

Visible light photocatalysis is a powerful tool to activate substrates or reagents and carry out chemical reactions under mild conditions with visible light as a reagent. Precision photochemistry is highly sought after for broad synthetic and biomedical applications, and critical for industrial applications that require multifunctional, high performance photocatalysts. There is a strong desire and effort to develop new photochemically active materials for use as organic photoredox catalysts to expand the number of available photocatalyst tools, and potentially open up new synthetic pathways. The benefits of using of simple organic dyes include reduced cost, toxicity, and overall environmental impact compared to traditional transition metal-based photocatalysts.

Photoredox catalysis is an important bond-forming synthetic methodology that continues to grow with increasing sophistication, organic reaction scope, and specificity. There is a considerable effort to shift away from expensive and toxic transition metal molecular catalysts (such as those using iridium and ruthenium) towards fully organic molecular catalysts. Organic photoredox catalysts can potentially provide a wider range of structural flexibility for tailoring solubility or improving charge transfer (CT) state characteristics in existing donor/acceptor photocatalyst systems.

### SUMMARY

In view of the foregoing, thiazolothiazole-based photocatalysts are described herein which, in some embodiments, exhibit advantageous redox potentials including positive excited state redox potentials for initiating coupling mechanisms involving one or more single electron transfers. In one aspect, thiazolothiazole-based compounds of Formula I are provided:

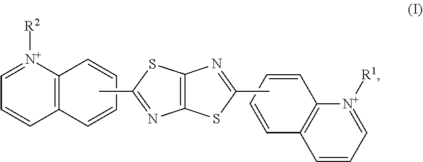

wherein R¹ and R² are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and −OR³, wherein R³ is selected from the group consisting of hydrogen, alkyl, alkenyl, and cycloalkyl. In some embodiments, compounds of Formula I are as follows:

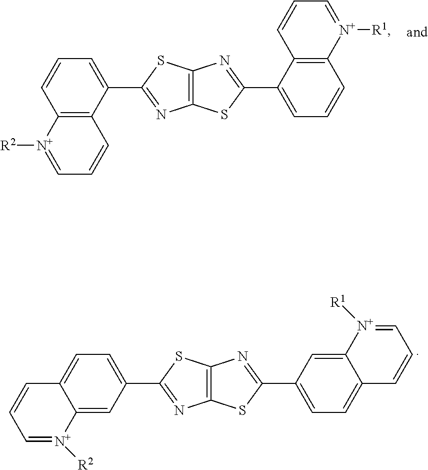

In another aspect, thiazolothiazole-based compounds of Formula II are provided:

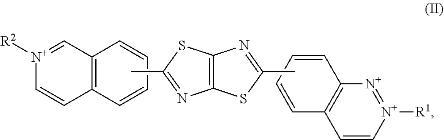

wherein R¹ and R² are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and −OR³, wherein R³ is selected from the group consisting of hydrogen, alkyl, alkenyl, and cycloalkyl. In some embodiments, compounds of Formula II are as follows:

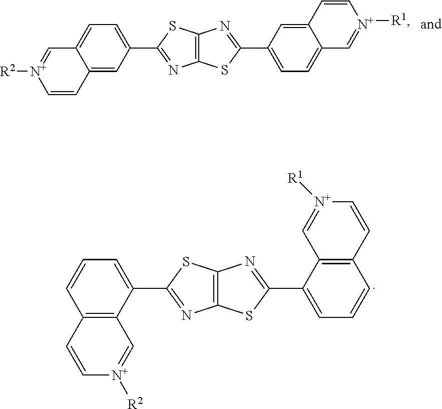

In another aspect, thiazolothiazole-based compounds of Formula III are provided:

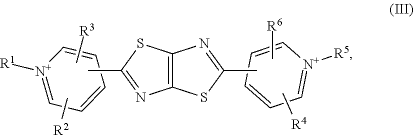

wherein R¹ and R⁵ are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and −OR⁷, wherein R⁷ is selected from the group consisting of hydrogen, alkyl, alkenyl, and cycloalkyl; and
wherein R² and R⁴ are independently selected from the group consisting of alkyl, alkenyl, cycloalkyl, heterocyclyl, halo, aryl, heteroaryl, alkylene-amine, alkylene-aryl, alkylene-heteroaryl, alkylene-heterocyclyl, and −OR⁸, wherein R⁸ is selected from the group consisting of hydrogen, alkyl, alkenyl, and cycloalkyl; and
wherein R³ and R⁶ are independently selected from the group consisting of hydrogen, alkyl, alkenyl, cycloalkyl, heterocyclyl, halo, aryl, heteroaryl, alkylene-amine, alkylene-aryl, alkylene-heteroaryl, alkylene-heterocyclyl, and —OR⁹, wherein R⁹ is selected from the group consisting of hydrogen, alkyl, alkenyl, and cycloalkyl. In some embodiments, compounds of Formula II are as follows:

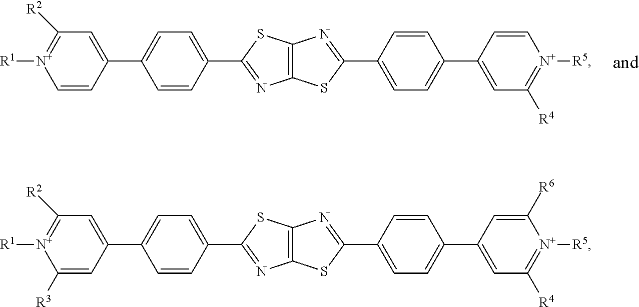

wherein R², R³, R⁴, and R⁶ are independently selected from the group consisting of alkyl, —OCH₃, and hydroxy;

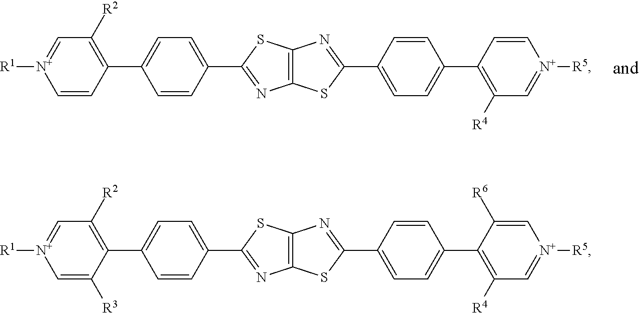

wherein R², R³, R⁴, and R⁶ are independently selected from the group consisting of —F and —Cl.

In another aspect, thiazolothiazole-based compounds of Formula IV are provided:

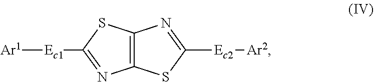

wherein Ar¹ and Ar² are independently selected from the group consisting of

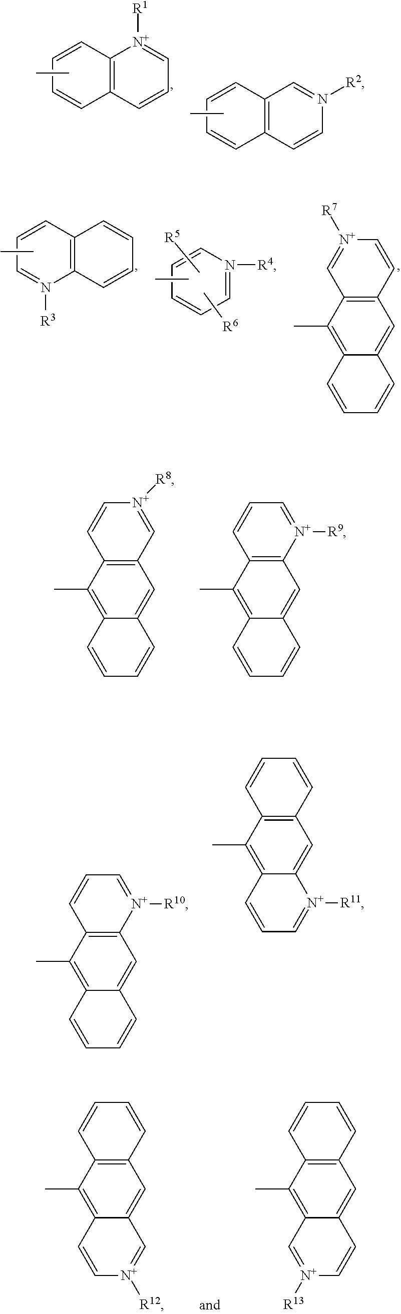

wherein R¹-R⁴ and R⁷-R¹³ are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and —OR¹⁴, wherein R¹⁴ is selected from the group consisting of hydrogen, alkyl, alkenyl, and cycloalkyl; and
wherein R⁵ and R⁶ are independently selected from the group consisting of hydrogen, alkyl, alkenyl, cycloalkyl, heterocyclyl, halo, aryl, heteroaryl, alkylene-amine, alkylene-aryl, alkylene-heteroaryl, alkylene-heterocyclyl, and —OR¹⁵, wherein R¹⁵ is selected from the group consisting of hydrogen, alkyl, alkenyl, and cycloalkyl; and
wherein E_{c1} and E_{c2} are independently selected from the group consisting of a direct bond, arylene, heteroarylene, alkenylene, alkenylene-arylene, alkenylene-heteroarylene, arylene-alkenylene, and heteroarylene-alkenylene. In some embodiments, the arylene or heteroarylene of E_{c1} is fused to Ar¹. Moreover, in some embodiments, the arylene or heteroarylene of E_{c2} is fused to Ar².

The conjugated moieties of E_{c1} and/or E_{c2} can be employed to extend the conjugation of the thiazolothiazole core, thereby facilitating absorption of longer wavelengths of visible radiation by the thiazolothiazole-based compounds. Such functionality can assist use of the thiazolothiazole-based compounds as photocatalysts in various coupling reactions. FIGS. 1-38 illustrate various compounds under Formula IV. R groups in FIGS. 1-38 can have any of the moieties recited above for compounds of Formula IV.

As described further herein, thiazolothiazole-based compounds of Formulas I-IV, in some embodiments, can serve as photocatalysts for various reactions including cross-coupling reactions. In some embodiments, for example, thiazolothiazole-based compounds of Formulas I-IV can serve as photocatalysts for alkylating substrates. A method of alkylation, in some embodiments, comprises irradiating a thiazolothiazole-based photocatalyst of any one of Formulas I-IV to place the photocatalyst in an excited state, and forming an alkyl radical via oxidation by the excited state thiazolothiazole-based photocatalyst. Oxidation by the thiazolothiazole-based photocatalyst places the photocatalyst in a reduced state. The alkyl radical subsequently attaches to an organic substrate to provide an alkylated organic substrate. The alkylated organic substrate is then reduced by thiazolothiazole-based photocatalyst, thereby providing the alkylated product and regenerating the thiazolothiazole-based photocatalyst. Reduction of the alkylated organic substrate can occur from the ground state of the thiazolothiazole-based photocatalyst.

These and other embodiments are further described in the following detailed description.

### BRIEF DESCRIPTION OF THE DRAWINGS

FIGS. 1-38 illustrate various compounds under Formula IV according to some embodiments.

FIG. 39 illustrates thiazolothiazole-based photocatalytic driven alkylation of amines according to some embodiments.

FIG. 40 provides a general photoredox catalytic reaction scheme and the TTz photophysical and redox properties.

FIG. 41 provides: a) Redox potentials of MePyTTz²⁺, isopropyl-BF₃K, and the N-centered radical intermediate, and b) TTz derivatives used as photoredox catalyst.

FIG. 42: provides: a) GCMS rate analysis of a photocatalytic reaction with (MePy)₂TTz²⁺ and isopropyl-BF₃K, and b) a first order fit of the rate of reaction, and c) the visual changing color of a reaction with [Bz₂TTz](PF₆)₂ and isopropyl-BF₃K.

FIG. 43: provides a) Light dependence investigation of the mechanism with (MePy)₂TTz and isopropyl BF₃K as time progresses and b) the average isolated yield and turnover number from ¹H NMR using trimethoxybenzene as an internal standard (n=2) with all TON values reported for 0.1 mol % catalyst loading.

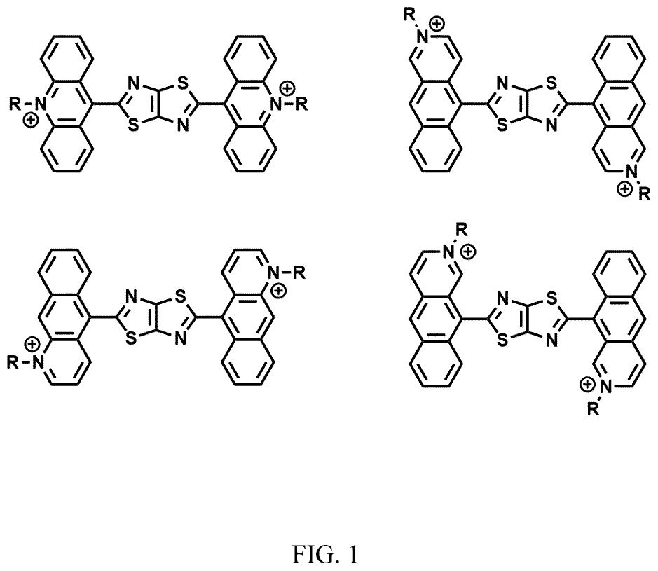

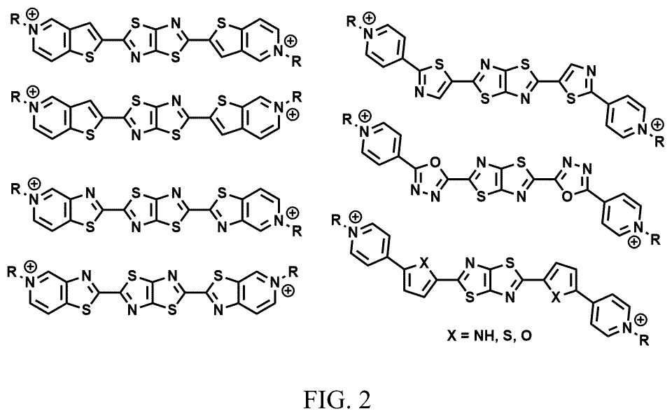

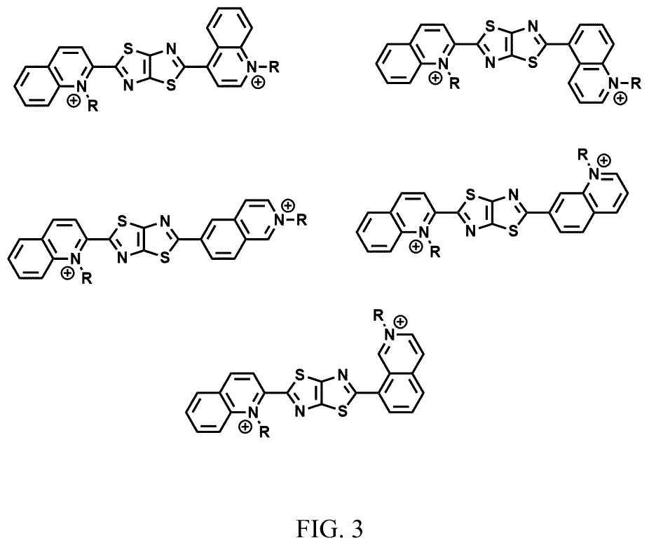

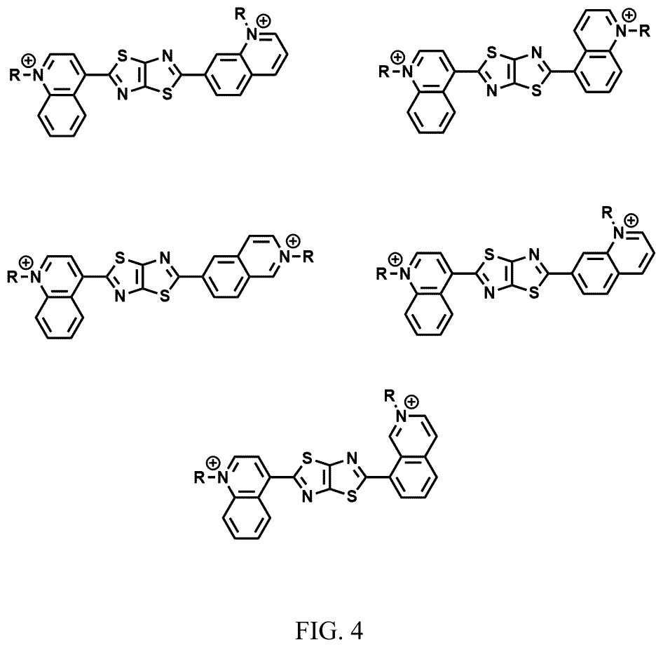

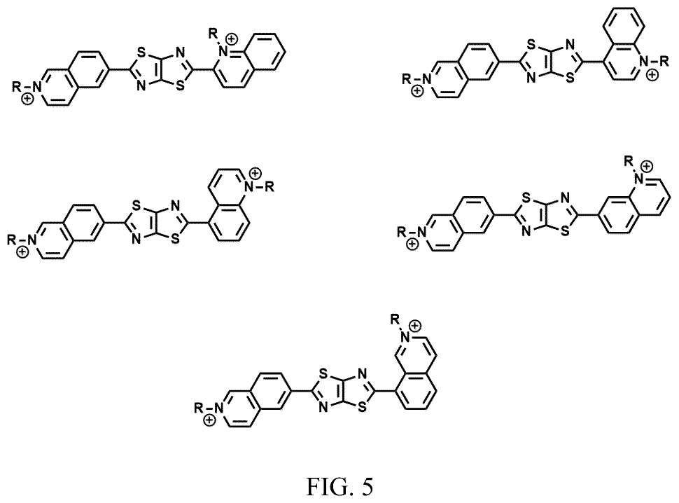

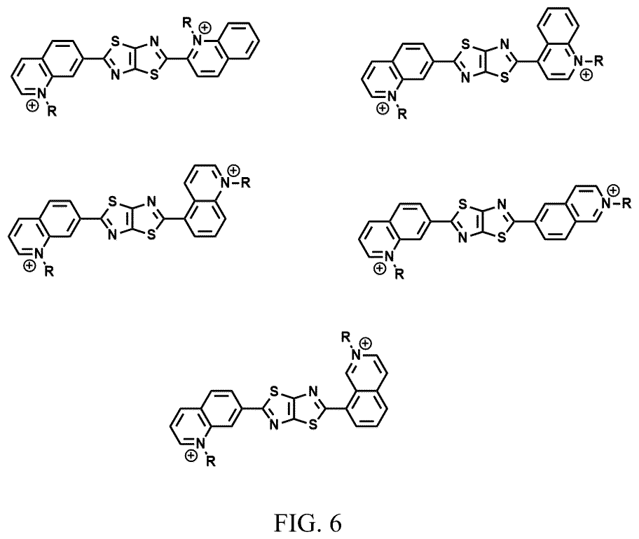

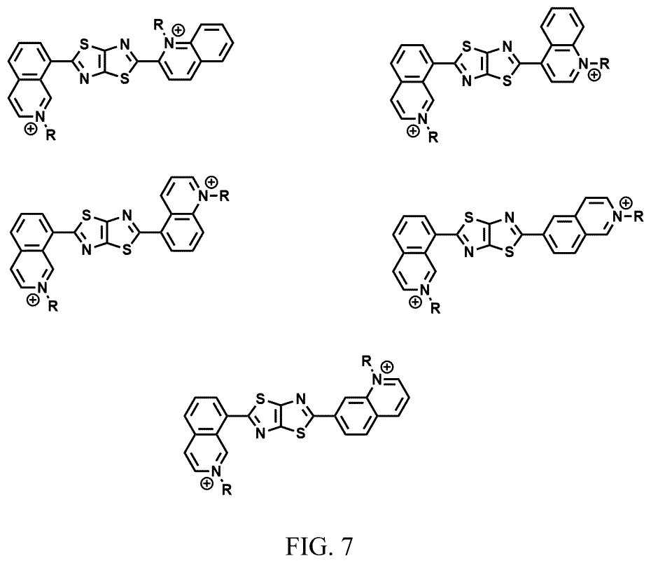

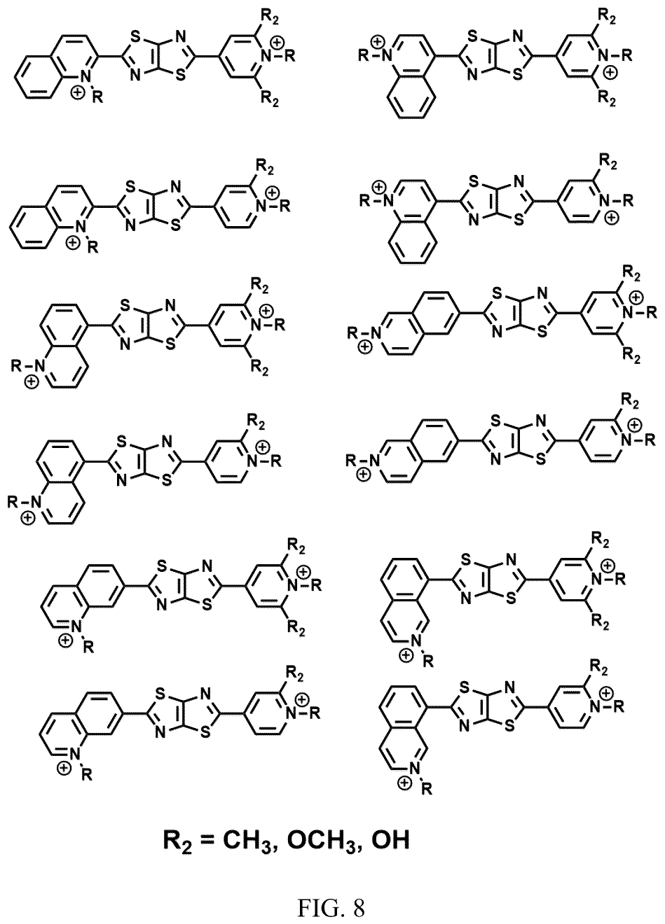

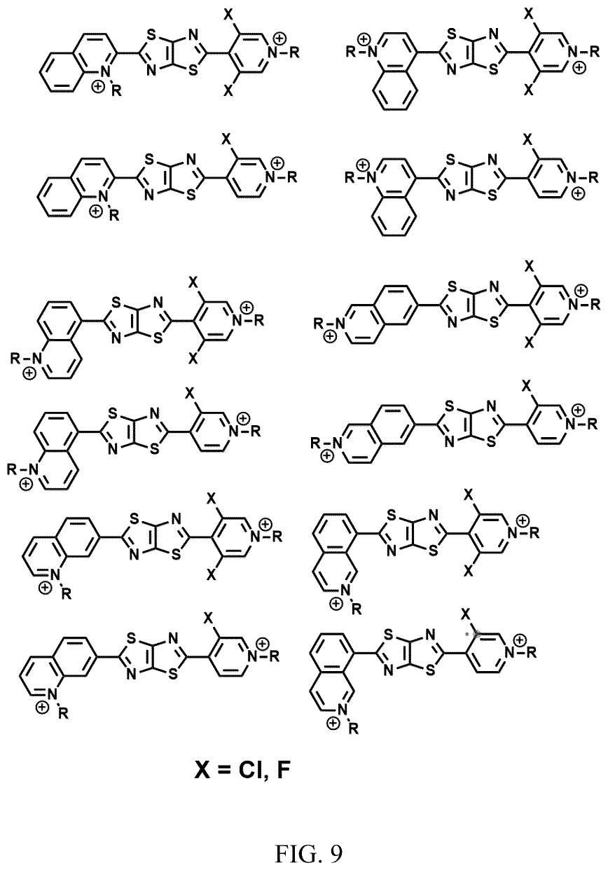

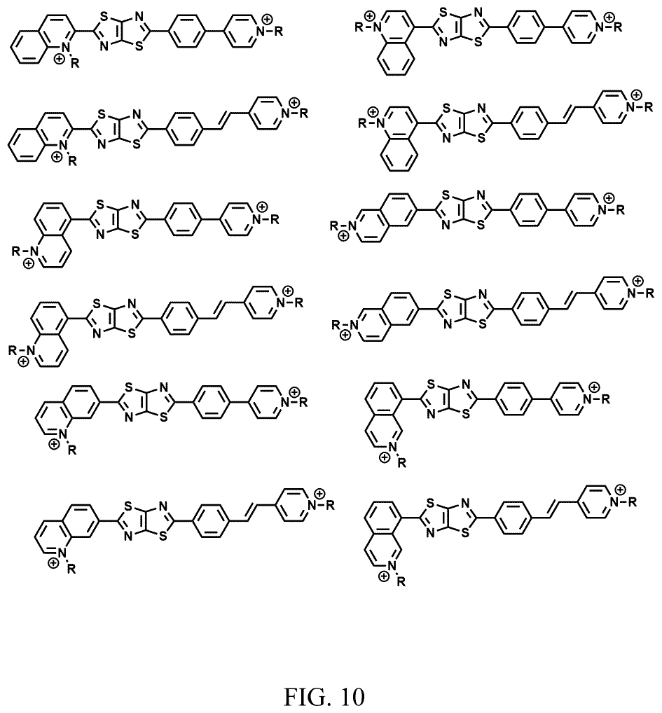

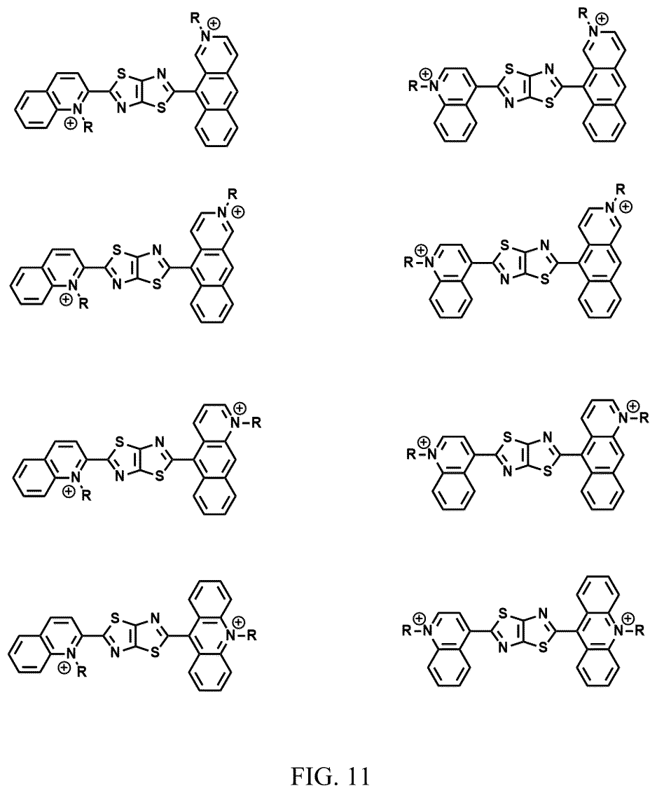

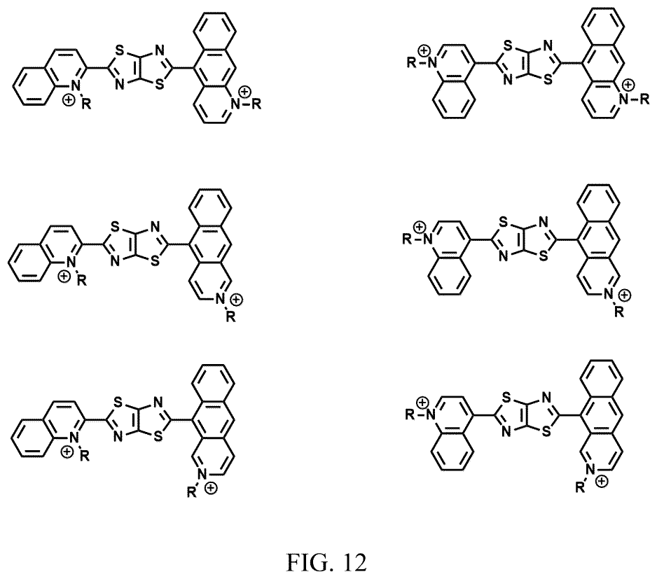

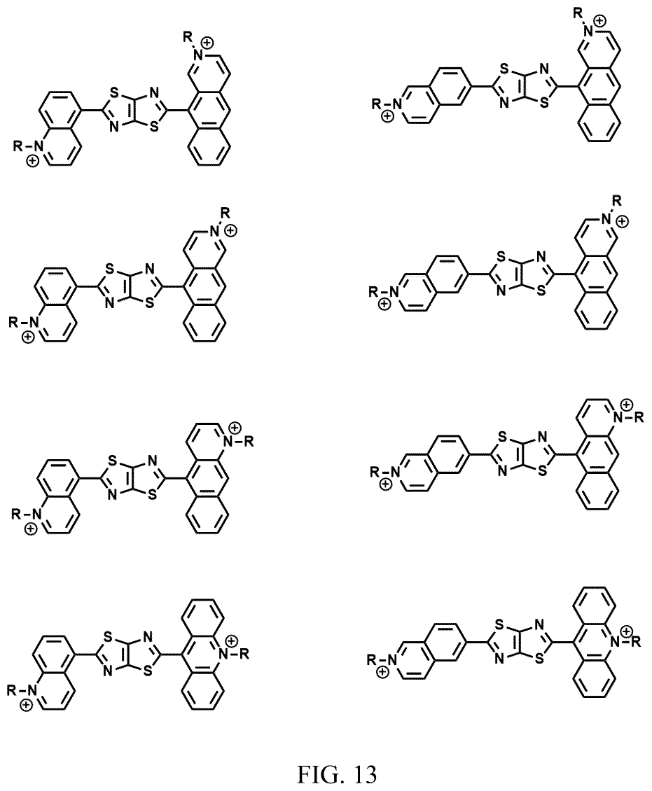

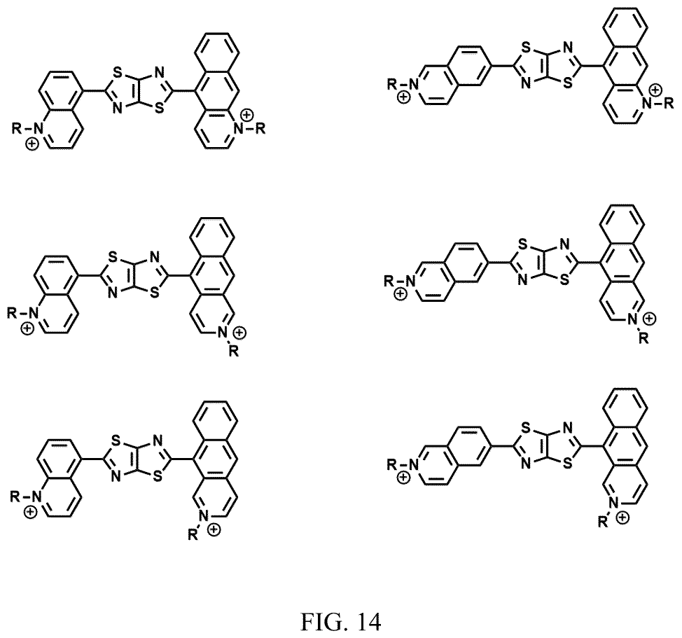

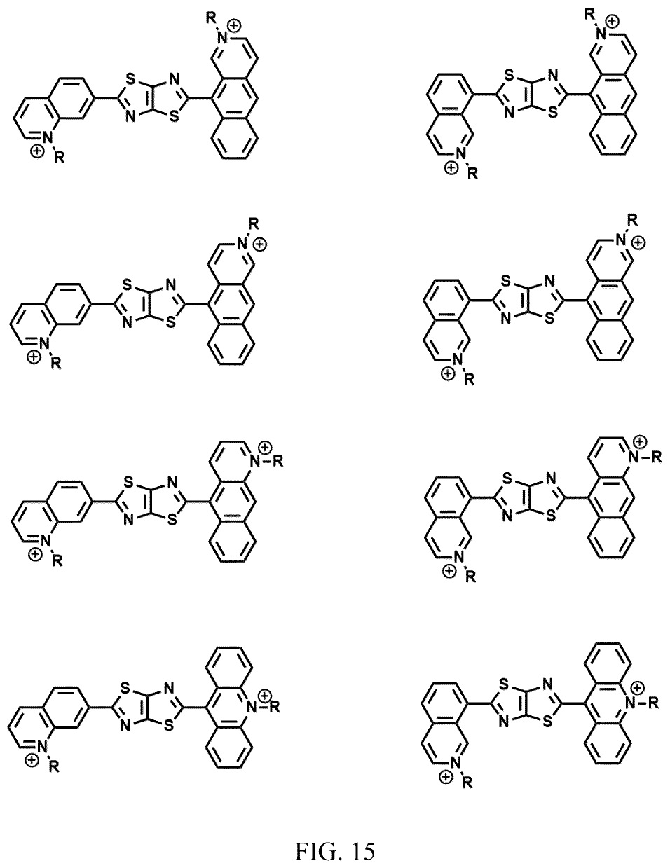

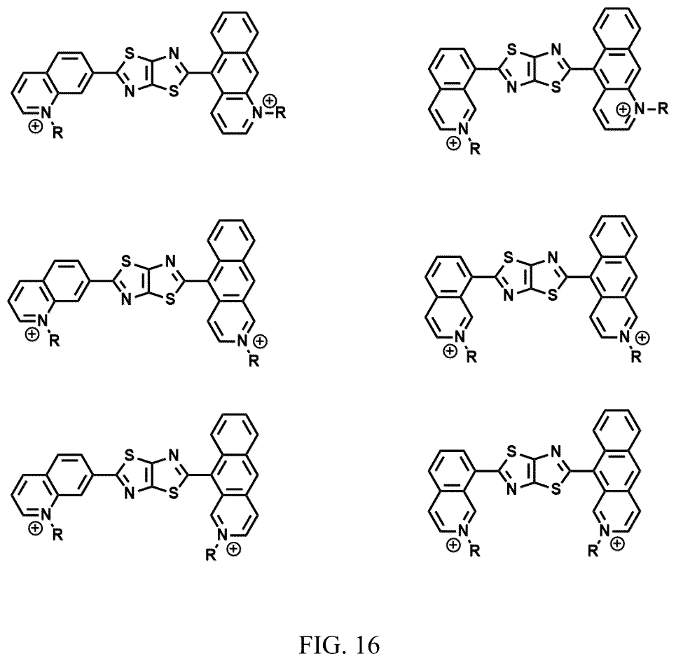

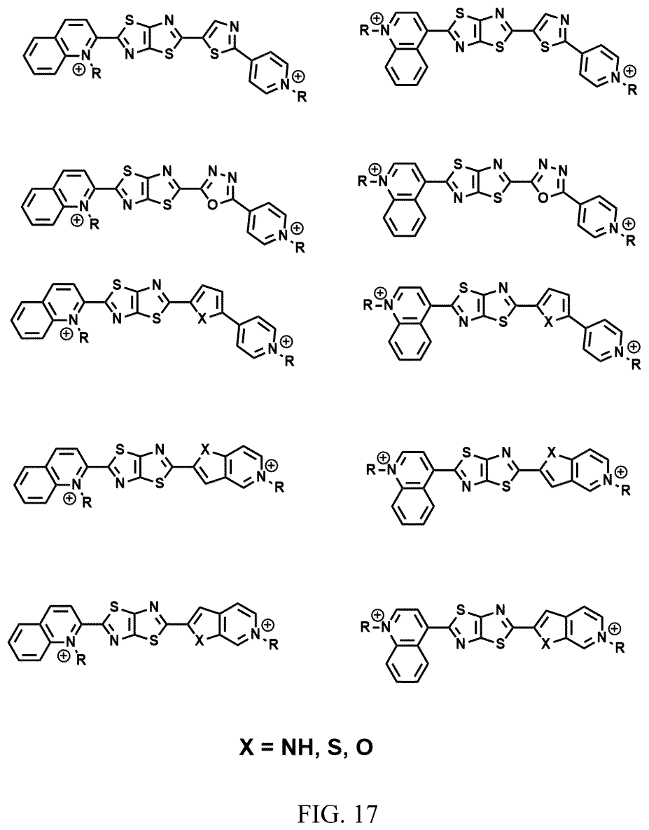

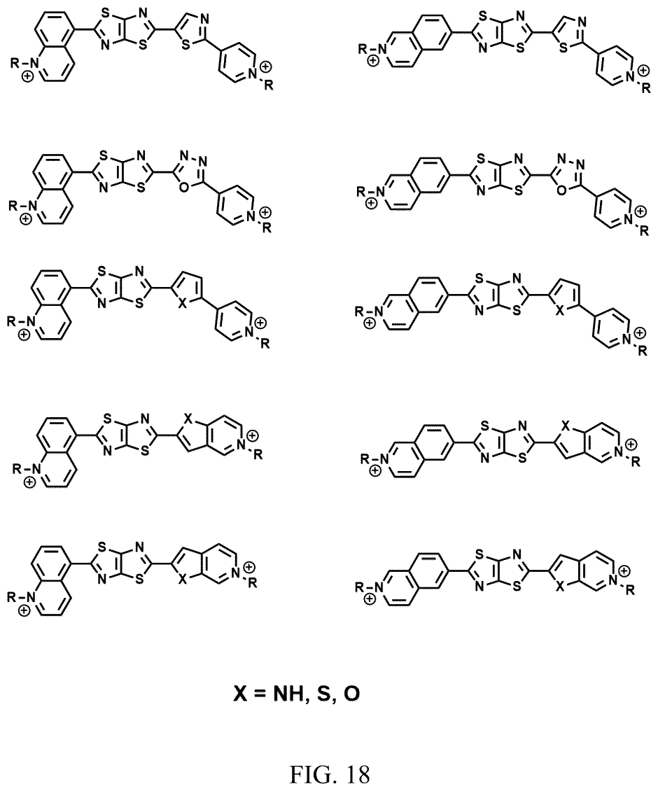

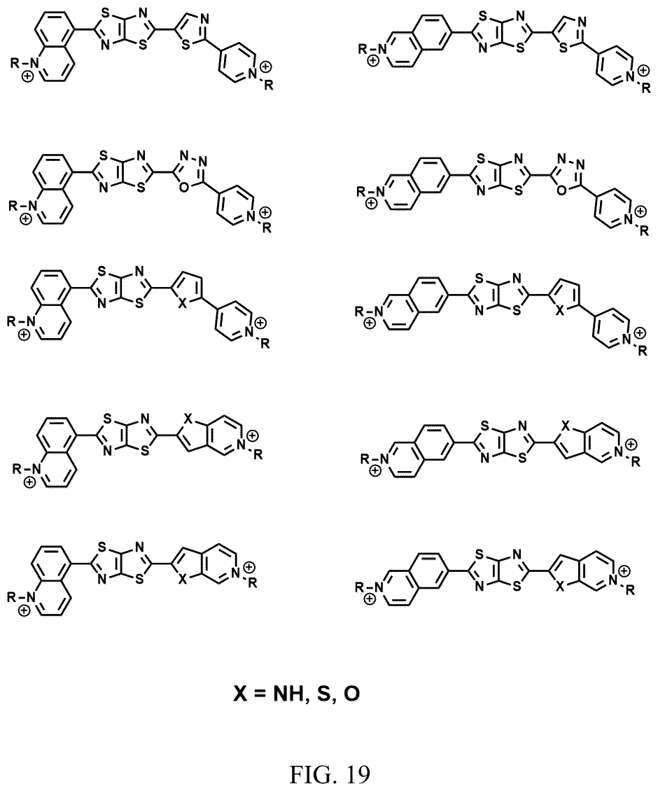

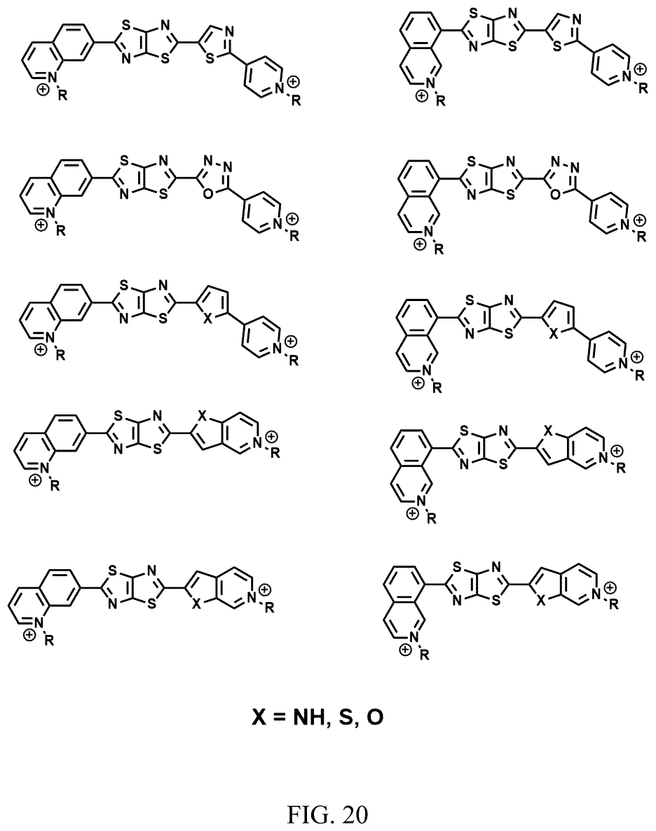

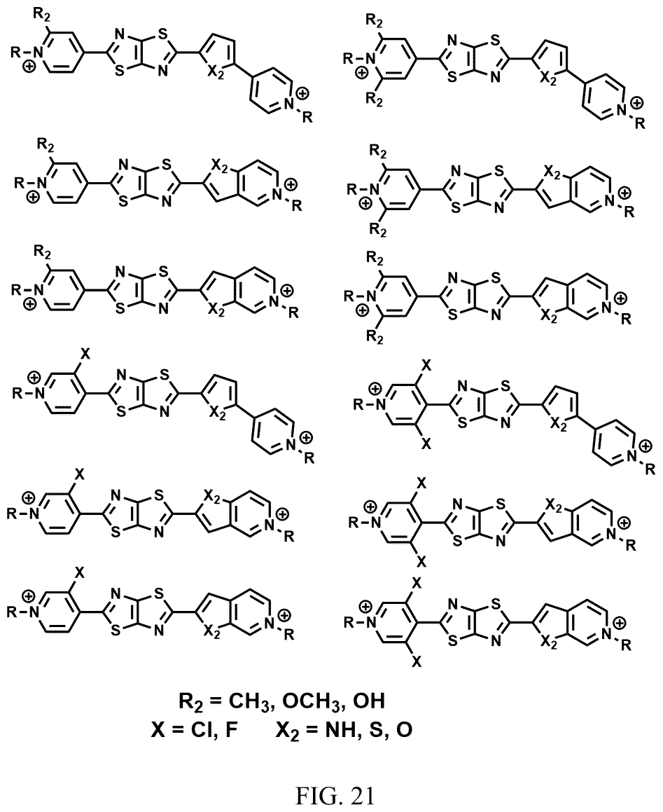

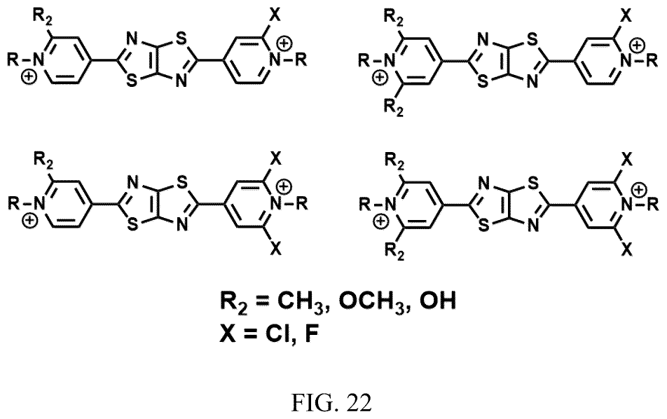

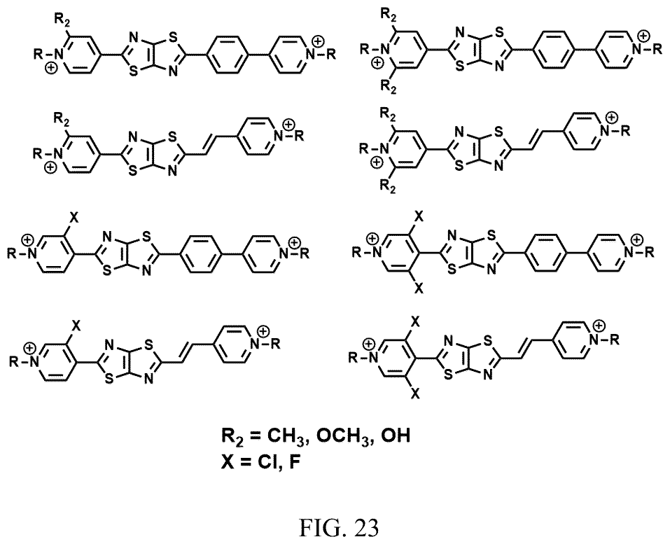

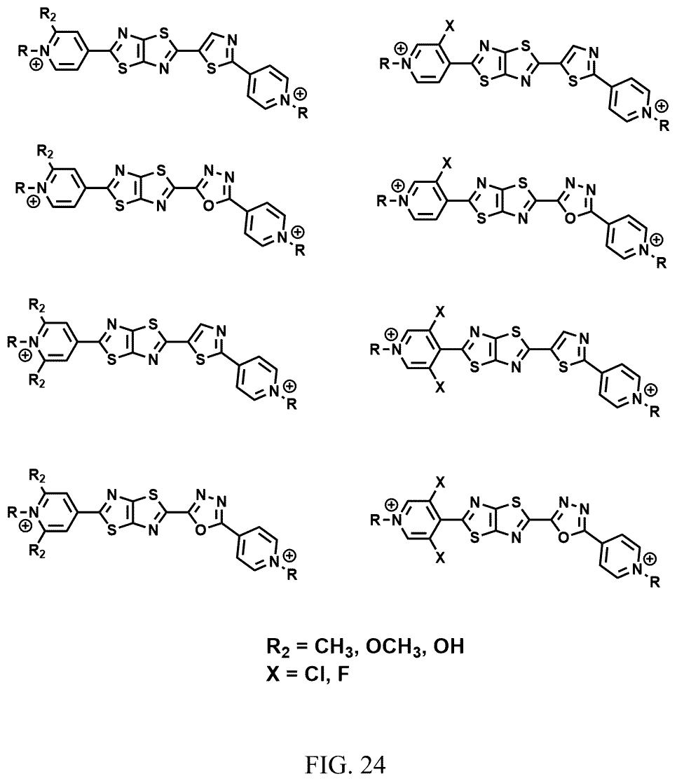

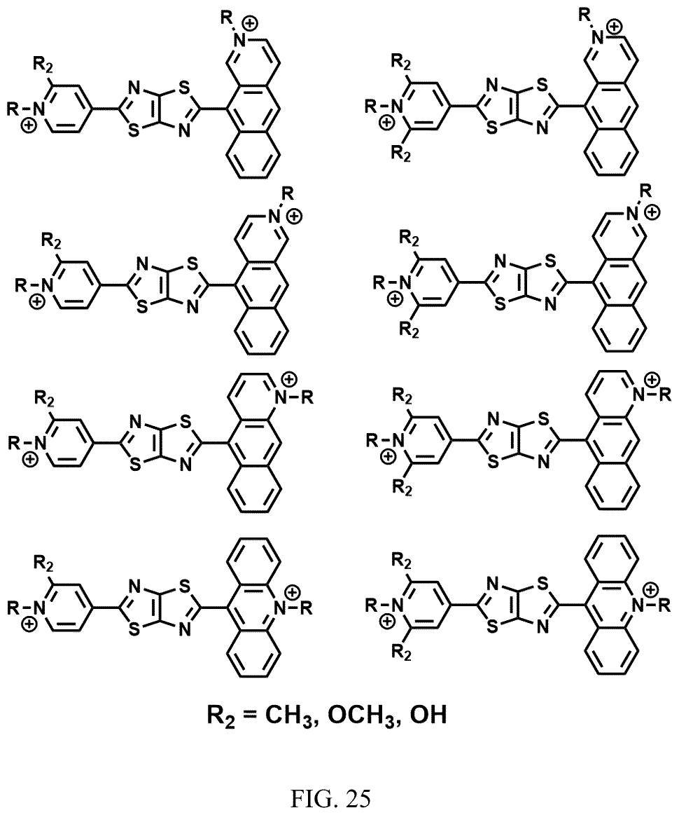

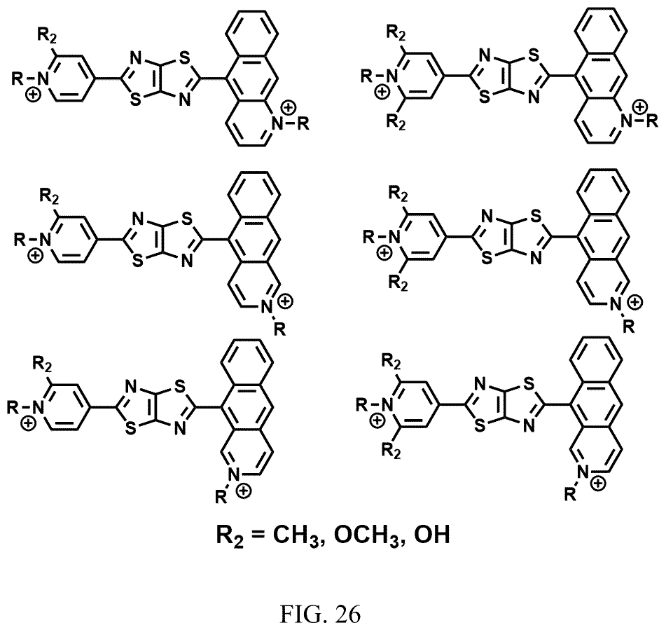

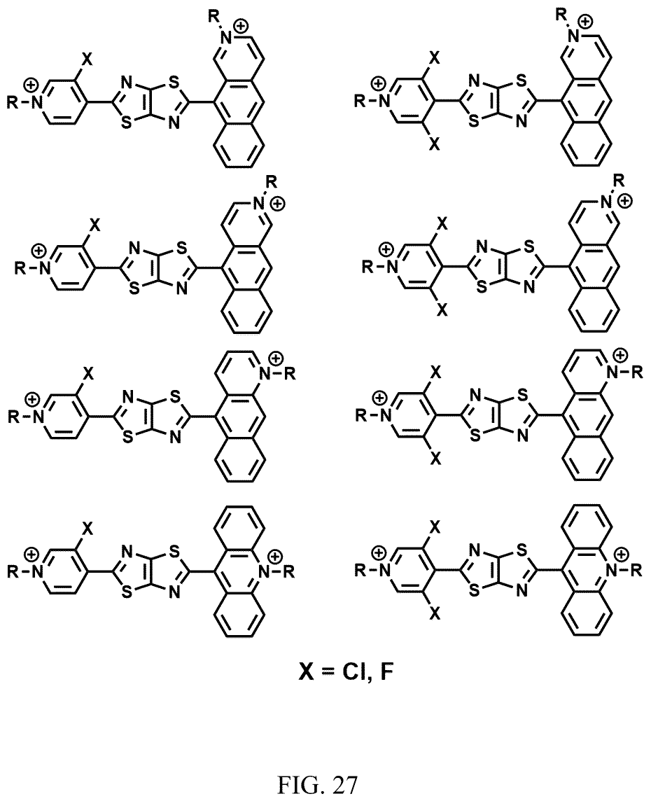

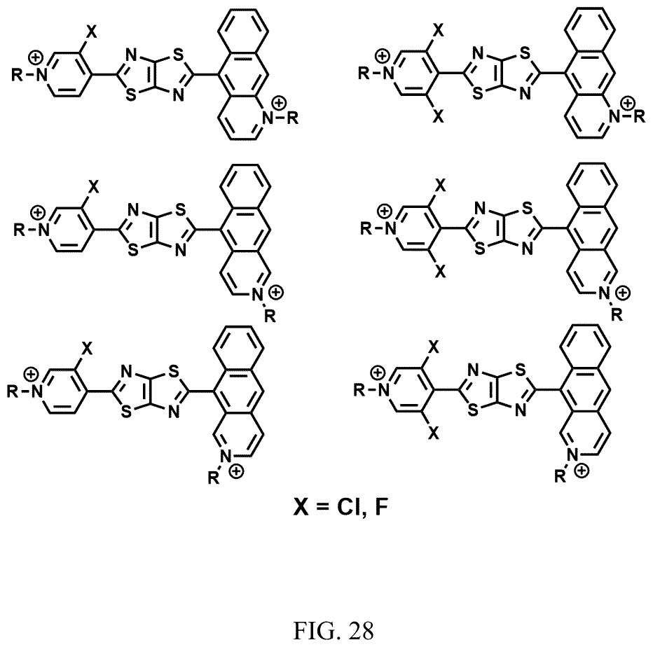

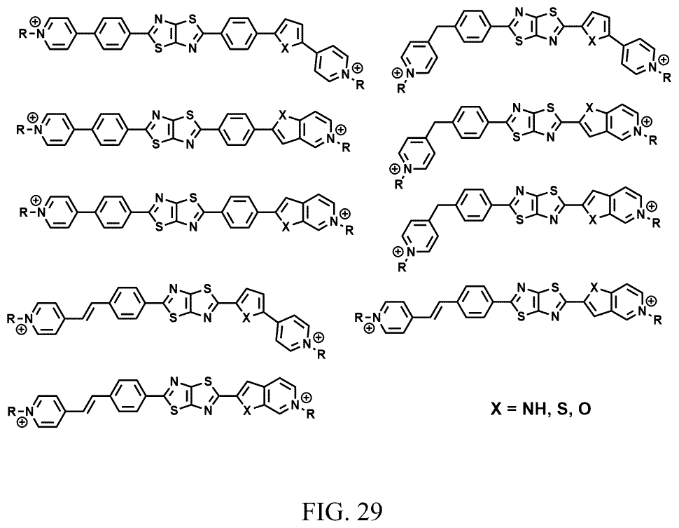

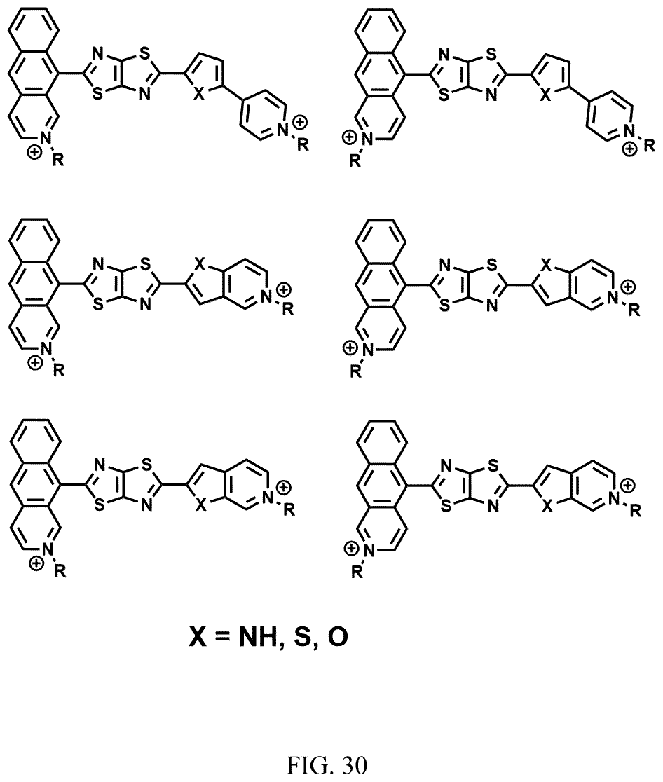

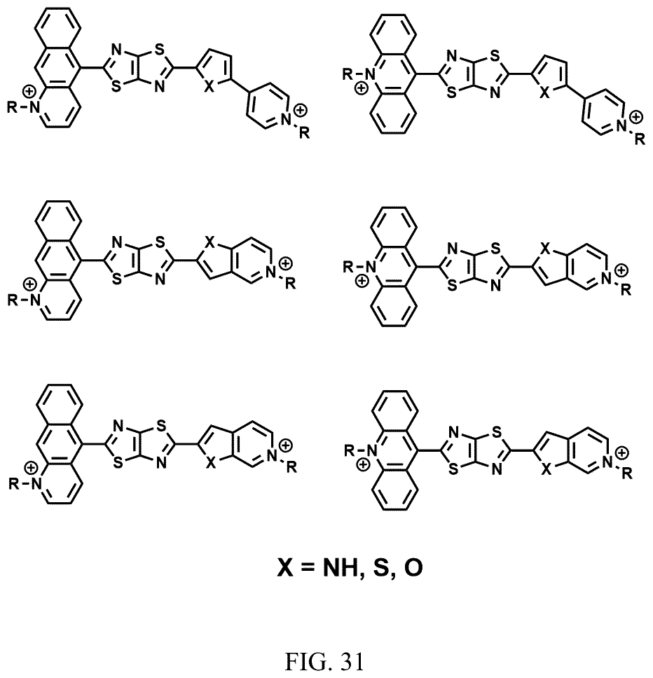

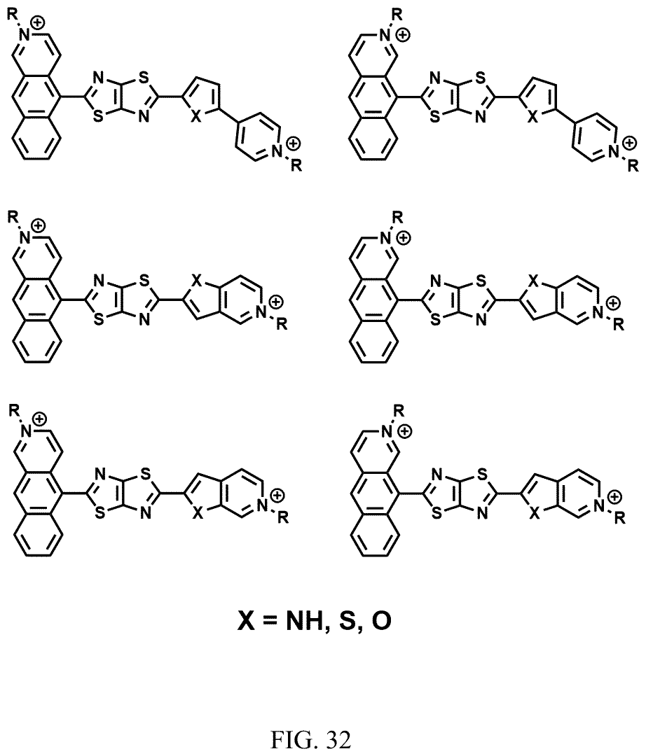

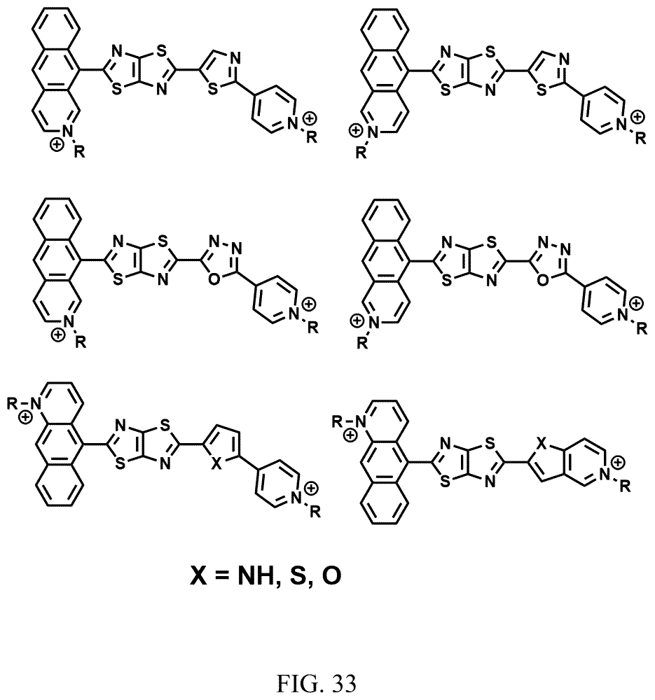

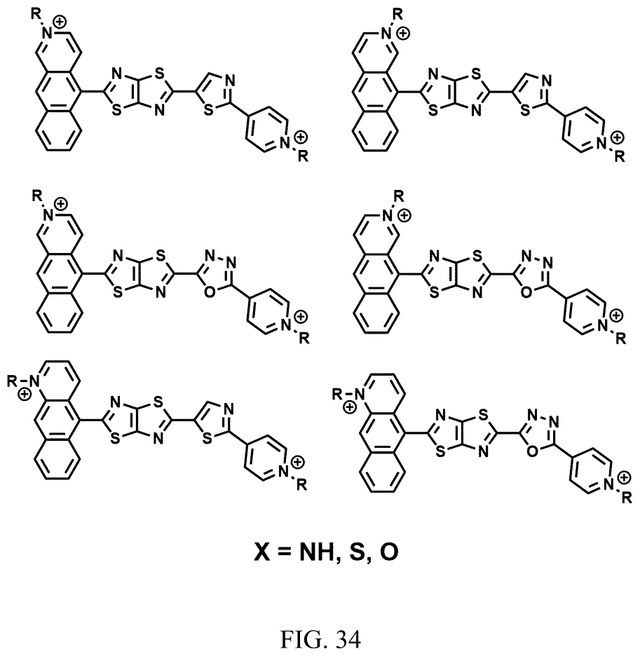

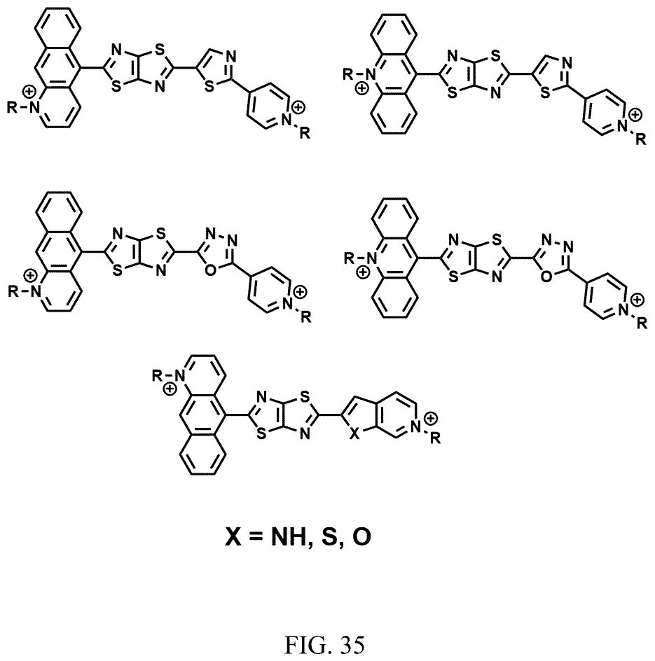

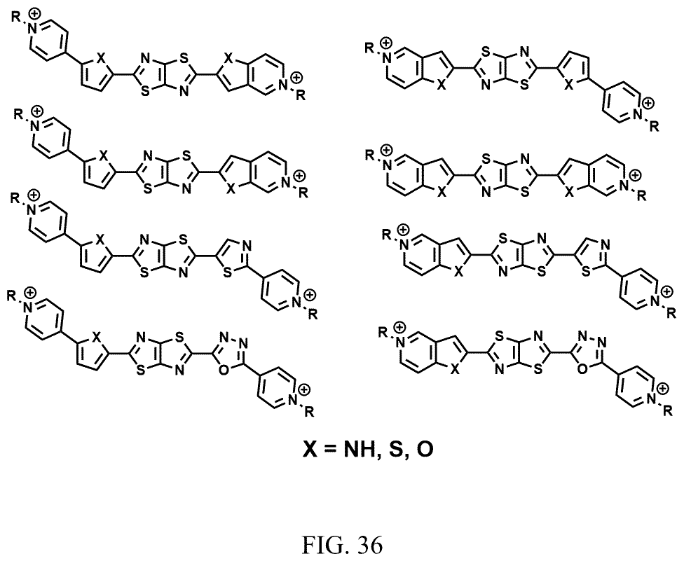

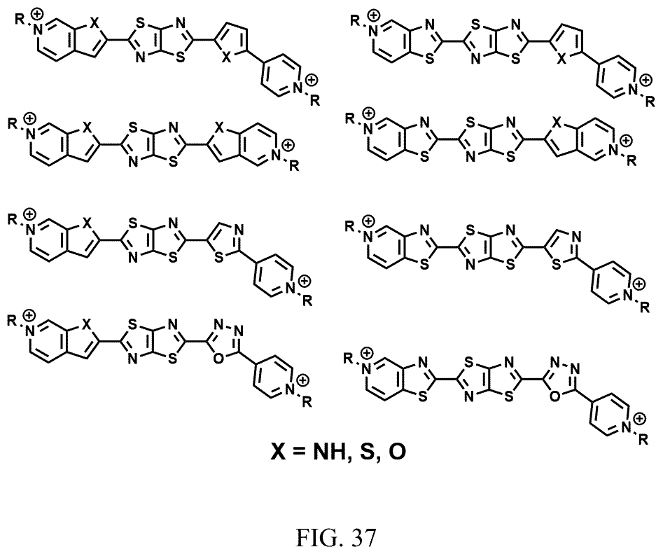

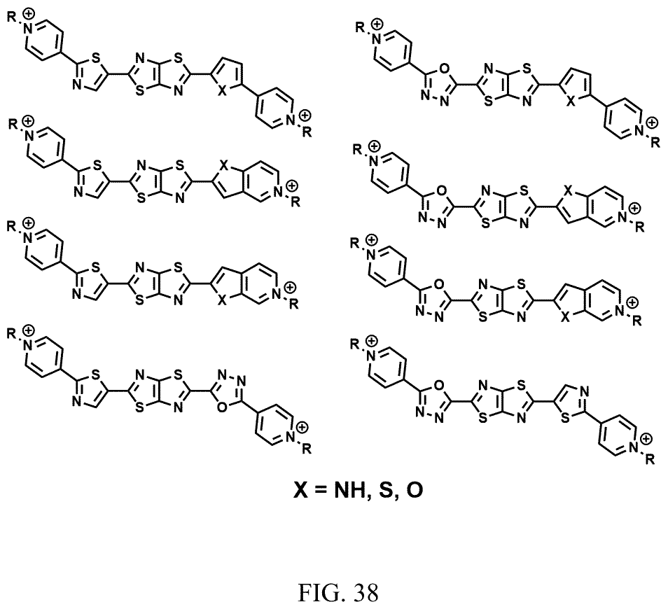

### DETAILED DESCRIPTION

Embodiments described herein can be understood more readily by reference to the following detailed description and examples and their previous and following descriptions. Elements, apparatus and methods described herein, however, are not limited to the specific embodiments presented in the detailed description and examples. It should be recognized that these embodiments are merely illustrative of the principles of the present invention. Numerous modifications and adaptations will be readily apparent to those of skill in the art without departing from the spirit and scope of the invention.

### Definitions

The term “alkyl” as used herein, alone or in combination, refers to a straight or branched saturated hydrocarbon group optionally substituted with one or more substituents. For example, an alkyl can be C₁-C₃₀ or C₁-C₁₈.

The term “alkenyl” as used herein, alone or in combination, refers to a straight or branched chain hydrocarbon group having at least one carbon-carbon double bond and optionally substituted with one or more substituents.

The term “aryl” as used herein, alone or in combination, refers to an aromatic monocyclic or multicyclic ring system optionally substituted with one or more ring substituents including, but not limited to, alkyl, alkoxy, hydroxy, carboxyl, and halo.

The term “heteroaryl” as used herein, alone or in combination, refers to an aromatic monocyclic or multicyclic ring system in which one or more of the ring atoms is an element other than carbon, such as nitrogen, boron, oxygen and/or sulfur. The monocyclic or multicyclic ring system may be optionally substituted with one or more ring substituents including, but not limited to, alkyl, alkoxy, hydroxy, carboxyl, and halo.

The term “heterocycle” as used herein, alone or in combination, refers to a mono- or multicyclic ring system in which one or more atoms of the ring system is an element other than carbon, such as boron, nitrogen, oxygen, and/or sulfur or phosphorus and wherein the ring system is optionally substituted with one or more ring substituents including, but not limited to, alkyl, alkoxy, hydroxy, carboxyl, and halo. The heterocyclic ring system may include aromatic and/or non-aromatic rings, including rings with one or more points of unsaturation.

The term “cycloalkyl” as used herein, alone or in combination, refers to a non-aromatic, mono- or multicyclic ring system optionally substituted with one or more ring substituents.

The term “halo” as used herein, alone or in combination, refers to elements of Group VIIA or Group 17 of the Periodic Table (halogens). Depending on chemical environment, halo can be in a neutral or anionic state.

Terms not specifically defined herein are given their normal meaning in the art.

Thiazolothiazole-based compounds of Formulas I-IV are described herein which, in some embodiments, exhibit advantageous electronic structure and redox potentials, including positive excited state redox potentials for initiating cross coupling mechanisms involving one or more single electron transfers. In some embodiments, thiazolothiazole-based compounds of Formulas I-IV are symmetric or asymmetric. Moreover, the thiazolothiazole-based compounds of Formulas I-IV can exhibit a difference (offset) between the highest occupied molecular orbital (HOMO) and the lowest unoccupied molecular orbital (LUMO) of at least 0.7 eV. Offset between the HOMO and LUMO can also have a value selected from Table I.

TABLE I
HOMO/LUMO Offset (eV)
1-4
2-4
1.5-3.5
2-3.5

Thiazolothiazole-based compounds of Formulas I-IV can exhibit positive excited-state redox potentials enabling oxidation of various substrates for initiating radical-based coupling reactions. The thiazolothiazole-based compounds of Formulas I-IV can be placed in the excited state via irradiation with light having wavelengths in the near UV and/or visible region of the electromagnetic spectrum. In some embodiments, thiazolothiazole-based compounds of Formulas I-IV have a peak absorption in the range of 380 nm to 700 nm.

As set forth in the structure of Formula IV, conjugation of the thiazolothiazole-based compounds can be extended via the alkenylene and/or aromatic moieties of E_{c1} and/or E_{c2}, thereby shifting the absorption characteristics of the compounds further into the visible region of the electromagnetic spectrum. Shifting the absorption characteristics further into the visible region can assist in use of the thiazolothiazole-based compounds as photocatalysts as lower energy radiation for photocatalyst activation can reduce the likelihood of generating unintended or competing reactive species in the reaction mixture.

When in the excited state, thiazolothiazole-based compounds of Formulas I-IV can exhibit reduction potentials for initiating radical-based coupling reactions. In some embodiments, thiazolothiazole-based compounds of Formulas I-IV have an excited-state reduction potential of at least +2 V relative to saturated calomel electrode (SCE). The excited-state reduction potential, in some embodiments, is +2V to +4V. As described further herein, these positive excited state reduction potentials permit thiazolothiazole-based compounds of Formulas I-IV to oxidize a number of organic substrates for radical formation. Oxidation of the substrate can place the thiazolothiazole-based compounds into a reduced state. The reduced excited state, in some embodiments, can also exhibit redox potentials operable to oxidize various organic substrates. In some embodiments, thiazolothiazole-based compounds of Formulas I-IV exhibit a reduced excited state redox potential of at least +1V. In some embodiments, the reduced excited state redox potential is +1V to +1.6V. Maintaining such positive reduced excited state redox potential values can enable thiazolothiazole-based compounds of Formulas I-IV to achieve high catalytic efficiencies at low loadings in the reaction mixture. In some embodiments, for example, the initial excited state and reduced excited state are each operable to oxidize the desired substrate in a coupling reaction. Accordingly, thiazolothiazole-based compounds of Formulas I-IV can be present in reaction mixtures at 0.05-1 mol. %, in some embodiments. Additionally, in some embodiments, thiazolothiazole-based compounds of Formulas I-IV can close the reaction cycle by reducing the alkylated substrate, thereby being regenerated to dicationic form for the next round of catalysis. Such reduction of the alkylated substrate can occur from the ground state of the photocatalyst.

In some embodiments, for example, thiazolothiazole-based compounds of Formulas I-IV can serve as photocatalysts for alkylating substrates. A method of alkylation, in some embodiments, comprises irradiating a thiazolothiazole-based photocatalyst of any one of Formulas I-IV to place the photocatalyst in an excited sate, and forming an alkyl radical via oxidation by the excited state thiazolothiazole-based photocatalyst. Oxidation by the thiazolothiazole-based photocatalyst places the photocatalyst in a reduced state. The alkyl radical subsequently attaches to an organic substrate to provide an alkylated organic substrate. The alkylated organic substrate is then reduced by the thiazolothiazole-based photocatalyst, thereby providing the alkylated product and regenerating the thiazolothiazole-based photocatalyst.

These and other embodiments are further illustrated in the following non-limiting examples.

### Example 1—Imine Alkylation With Thiazolothiazole-Based Photocatalyst

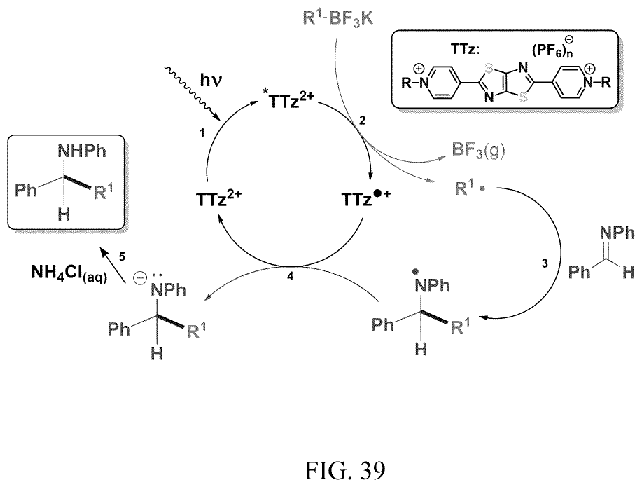

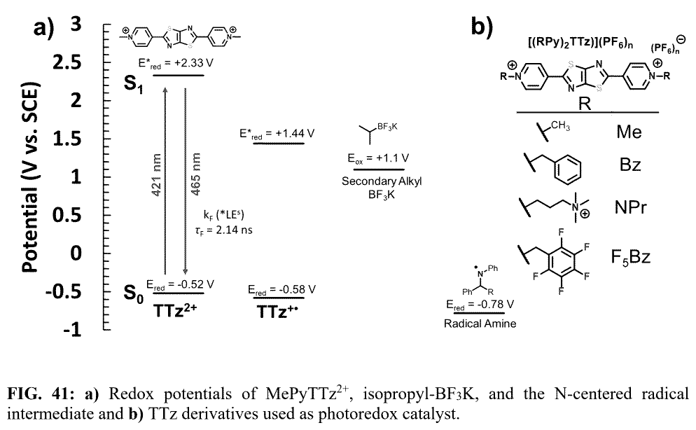

FIG. 39 illustrates the photochemically driven, redox-neutral imine alkylation reaction using the thiazolothiazole dipyridinium (TTz²⁺) photocatalyst used in this example. The excited-state redox potential of TTz²⁺ (S₀ to S₁ transition) (FIG. 41) is sufficiently large enough to oxidize the R-BF₃K substrate, via a single-electron-transfer (SET), to generate R^{⋅} that can react with the imine to form the N-centered radical. The amide ion is proposed to be formed by subsequent reaction with the TTz^{⋅+} to reform the TTz²⁺ with the product amine forming after workup/protonation. The reaction is driven by the strongly oxidizing photoexcited state of the photocatalyst whose excited-state lifetime is sufficient to begin generating alkyl radicals from the R-BF₃K substrates via C—B bond cleavage.

### General Photoredox Catalysis

Imine (0.25 mmol), R-BF₃K (0.33 mmol), and (R′)₂TTz²⁺ (5.8 μmol, 0.1 mol %) were added to a 2-dram vial with a stir bar. The vial was taken into the glovebox (N₂ environment) where 5 mL of anhydrous DCM were added. The vial was sealed, sonicated, then illuminated for 48 h at 450 nm or 420 nm with LED lights (using a Penn PhD Photoreactor M2) while stirred under anaerobic conditions. When stopped, 1 drop of the reaction mixture was diluted with 1 mL of ether and conversion of imine was determined via GCMS. The reaction mixture was quenched with NH₄SO₄ (aq), the organic layer separated, and the remaining aqueous layer was extracted with CH₂Cl₂ (3×10 ml). The combined organic extracts were evaporated and a known amount of 1,3,5-trimethoxybenzene (˜20 mg) was added as an internal standard. All the components were then added to an NMR tube with CDCl₃. A ¹H-NMR was obtained and integrations of the 1,3,5-trimethoxybenzene are compared to the product to determine the isolated yield.

### Reaction Rate Analysis

The rate of the reaction was tracked by GCMS. An additional 100 μL of decane was added to a reaction as an internal standard in the glovebox. At desired time intervals, the reaction was stopped, taken into the glove box and an aliquot of 20 μL was obtained. The reaction was resealed and placed back into the photoreactor to continue. The aliquot was quenched with 50 μL of methanol, diluted with 1 mL of ether and 1.5 μL was injected into the GCMS. The method was an inlet at 200° C., with the column held at 50° C. for 3 min then ramped 20° C./min to 250° C. and held for 5 min. To lower uncertainty, an extracted ion chromatogram was made with 180 M⁺, 182 M⁺, and 57 M⁺, the max ion peaks, and the area recorded for the imine reactant (r_{f}=11.30 min), amine product (r_{f} in supplemental), and decane (r_{f}=6.10 min), respectively. Once the reaction was complete, the isolated yield was determined from the internal standard in ¹H-NMR and used to determine the relative yield throughout the reaction.

### Photocatalytic Results

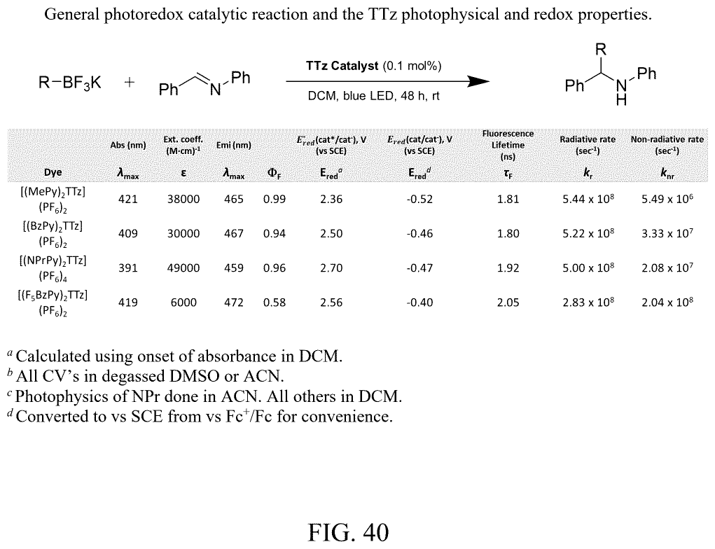

Typical of dipyridinium TTzs, the derivatives in this example have a max absorbance (λ_{abs}) ranging from 391 to 421 nm in DCM, with max emissions (λₑₘᵢ) from 453 to 472 nm. Thus, they have a large potential window capable of photoredox catalysis. The TTz derivatives have relatively high fluorescence QYs (Φ_{F}=0.58 to 0.99) and short fluorescent lifetimes (τ_{f}=1.73 to 2.05 ns) (FIG. 40). The short τ_{f} and poor solubility in DCM made obtaining a Stern-Volmer (SV) plot difficult but with a steady state of illumination, TTz²⁺ becomes reduced and isopropyl-BF₃K quenches the catalyst. In comparison, the starting imine did not quench fluorescence providing evidence that the TTz oxidizing the R-BF₃K takes place before other major steps in the catalytic cycle, similar to when an iridium photocatalyst was used.

Redox values were used to determine the spontaneity for the individual steps in the catalytic cycle. The E_{1/2} values between the individual TTzs were similar therefore Me₂TTz is used as an example. Using a simple equation (1) derived from the Gibbs energy of photoinduced electron transfer equation,⁷ the excited state catalytic driving potential is calculated.

$${E}_{r e d}^{*} ( {cat}^{*} / {cat}^{-} ) = {E}_{r e d} ( cat / {cat}^{-} ) + {E}_{0 , 0} \qquad ( 1 )$$

Where E*_{red} is the excited state reduction potential, E_{red} is the ground state reduction potential and E_{0,0} is the energy minimum vibrational state of the excited state as estimated by the onset of the absorbance. Then using the standard redox equation (2), spontaneity is determined.

$${E}_{r x n}^{◦} = {E}_{red}^{◦} + {E}_{ox}^{◦} \qquad ( 2 )$$

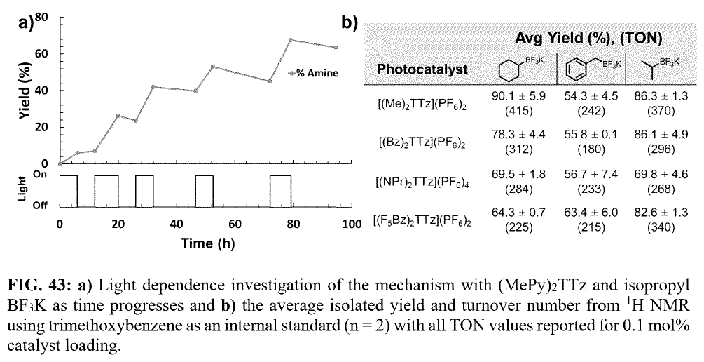

Initially, when the TTz²⁺ is in the ground state, oxidation of the R-BF₃K isn't favorable (Eᵣₓₙ=−1.62 V). However, the oxidation becomes highly favorable upon excitation (Eᵣₓₙ=1.23 V). In step four, the TTz^{⋅+} reducing the N-centered radical is slightly unfavorable (Eᵣₓₙ=−0.20 V), yet a high yield is still obtained (FIG. 43b). Previous spectroelectrochemistry of pyridinium TTzs showed decreased emission when reduced due to radical quenching, but a constant large molar absorptivity.¹¹ Given this and the redox potential, the excited TTz^{⋅+} shows promise at being able to reduce an additional R-BF₃K substrate (Eᵣₓₙ=0.34 V) (FIG. 41a).

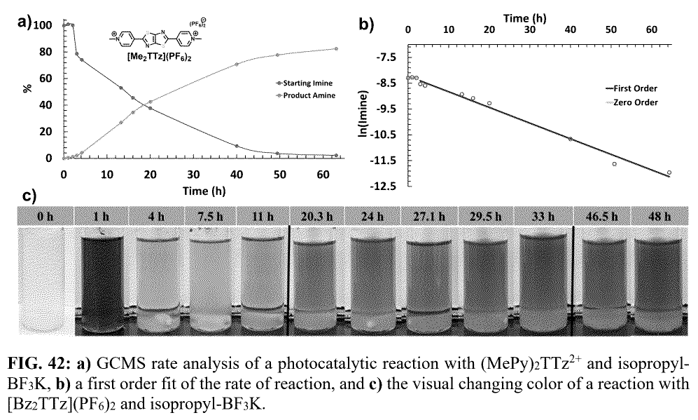

The rate of the reaction was initially monitored to determine the optimal runtime but visually and then spectroscopically, the TTz^{⋅+} was strongly observed in the first hour but faded below detection limits (FIG. 42c). The TTz is speculated to oligomerize with itself or other free radicals, like the R^{⋅} or radical DCM, in solution. DCM reacting with the imine can form a byproduct which is observable by GCMS but trace detection of catalyst byproducts proved difficult. Further methods will be examined in later works. GCMS analysis was conducted to track the overall reaction progress and for analyzing reaction mechanisms. Initially there is no decrease in imine (˜1 h) followed by a sharp decrease into a linear relationship for the rest of the reaction. When plotting the imine concentration on a natural log, the reaction initially shows a zero-order rate. This stagnation in reaction time demonstrates the rate-limiting step is the excited TTz²⁺ oxidizing the R-BF₃K due to the fast rate of emission (k_{f}=1.81 ns). Once the unknown competitive pathway is exhausted, the reaction shows a pseudo first-order reaction with respect to the imine. The amine product then has an equal rate of formation as that of the imine disappearance, showing the rate of TTz^{⋅+} reducing the N-centered radical to an amide was a non-rate limiting step. To further investigate the mechanism, a light-dependent experiment was conducted providing evidence for a closed-cycle mechanism instead of a radical chain process (FIG. 43a).

The catalyst loading needed was much lower (0.1 mol %) than common iridium and acridinium PCs (5 and 2.5 mol %, respectively). Quantitatively, the low mol % increased the turnover number (TON) of reactions ((MePy)₂TTz, TON_{avg}=332) but showed a lower turnover frequency (TOF). A wide range of catalyst loadings were tested (5 to 0.025 mol %) before determining that 0.1 mol % produced the highest yield within 48 h. Using a serial dilution, reactions with TTz as low as 0.025 mol % were run and demonstrated a large TON ((MePy)₂TTz at 0.025 mol %; yield=43.7%, TON=791). The high TON with a low mol % is characteristic of the strong photostability of the TTz derivatives.

All TTzs after ion exchanging with hexafluorophosphate showed increased but still poor solubility in DCM. The TTzs have comparable yields (FIG. 43b) except for the NPr₂TTz, which is likely due to the lower solubility of NPr₂TTz, with four cations, where the photophysical properties could not be obtained in DCM. (F₅Bz)₂TTz was synthesized to see if an increase in solubility would increase yield and/or rate. Despite increased solubility, neither an increased rate nor yield were observed with (F₅Bz)₂TTz, however, comparable yields were observed despite (F₅Bz)₂TTz having the lowest fluorescence QY and extinction coefficient (ε) of TTzs in this work. Like previous pyridinium TTzs, (F₅Bz)₂TTz was confirmed to have two single electron transfers with square wave voltammetry. Overall, large yields were obtained contrary to the poor solubility and short τ_{f}, typically limiting the ability of the catalytic cycle to turn over.

In summary of this example, dipyridinium TTz organic photocatalysts were synthesized with large QYs (Φ=0.58 to 0.99) in aqueous and organic solvents and compatible redox ranges for effective photooxidation of R-BF₃K and radical imine coupling to form an α-arylamine (isolated yields˜70-90%). The TTz photocatalysts have a large TON (TON, 400-700) due to the catalyst loading (0.1 mol %) for driving the coupling reaction. Despite the short fluorescent lifetimes that limit the product yield quantum efficiency, the TTz photocatalyst can be stably illuminated for 48 h and produce yields higher than iridium photocatalysts. Using GCMS, it was determined that a more soluble TTz did not effectively increase the rate or yield of the reaction and provided evidence that the short lifetime does limit the initial step of the reaction, going from a zero-order to a psuedo first-order. Lastly, the singly reduced state of the photocatalyst was spectroscopically observed within the first hour providing strong evidence of it's presence in the proposed photocatalytic mechanism and the ability for the singly reduced TTz to turnover in the catalytic cycle.

### Materials and Methods

Dithiooxamide, 4-pyridinecarboxaldehyde, benzyl bromide, (3-bromopropyl)-trimethylammonium bromide, ammonium hexafluorophosphate (NH₄PF₆), toluene, dimethyl sulfoxide (DMSO), acetonitrile (ACN), methanol, hexanes, hydrochloric acid, dimethyl formamide (DMF) and dichloromethane (DCM) were all purchased from Sigma-Aldrich or Baker Scientific. R-BF₃K reagents were purchased from Frontier Specialty Chemicals. Unless otherwise stated, all reagents were used without further modification. Py₂TTz, [(MePy)₂TTz](PF₆)₂, [(NPr)₂TTz](PF₆)₄, and [(BzPy)₂TTz](PF₆)₂ were all synthesized similar to previous with any modification in the supplemental.^{9-10, 12 1}H and ¹³C-NMR measurements were taken using a JEOL 500 MHz or 300 MHz NMR. High resolution mass spectra were obtained using a Voyager Matrix Assisted Laser Desorption/Ionization Time-of-Flight (MALDI-TOF) mass spectrometer using 1,8,9-Anthracenetriol as a matrix. Gamry Reference 600 potentiostat, Cary 300 UV-Vis spectrophotometer, Shimadzu RF-5301PC spectrofluorophotometer and a Jobin Yvon-Spex Fluorolog spectrofluorometer were used for spectra collection, fluorescence lifetime and quantum yield, and molar extinction coefficient determination. All lifetimes reported have a χ² less than 1.2. Gas chromatography was obtained on an Agilent 5975C VL MSD with triple axis detector and a DB-1MS column (L=30 m, Dia=0.250 mm, Film=0.25 μm). High-performance liquid-chromatography (HPLC) was obtained on a Thermo Accela HPLC system used with a low-pressure mixing quaternary pump, vacuum degasser, autosampler with a 10 μL sample loop, temperature-controlled column, a photodiode array detector (PDA), and a thermos accucore C4 column (10 cm length, 2.1 mm ID, 3 μm particle).

### Cyclic Voltammetry (CV)

All electrochemical experiments were carried out in Argon purged DMSO or ACN with 0.5 M tetrabutylammonium hexafluorophosphate as supporting electrolyte solutions using a

Gamry Reference 600 potentiostat and a three-electrode setup. A 3 mm, PEEK-encased, glassy carbon or Pt disk electrode was used as the working electrode. A platinum foil was used as the counter electrode. The reference electrode was a Ag/AgNO₃ quasi-reference electrode versus ferrocene. The redox values were reported against Fc/Fc⁺ but converted to vs SCE for convenience. Square wave voltammetry was also performed in acetonitrile using a pulse size of 15 mV and a frequency of 10 Hz.

Synthesis of N,N′-di(pentafluorobenzyl) 2,5-bis(4-pyridinium)thiazolo[5,4-d]thiazole dichloride [(F₅BzPy)₂TTz⁺⁴](PF₆)₂. PyTTz (0.0544 g, 0.184 mmol) and 2,3,4,5,6-pentafluorobenzyl bromide (1.5 mL, 9.93 mmol) were placed in a 2-neck 50 mL RBF and refluxed at 130° C. for 72 h in a nitrogen environment. The bright yellow reaction mixture was then cooled to RT. The reaction mixture was dissolved in water and washed twice with 10 mL of DCM. The aqueous phase was collected separately and approx. 1 g of NH₄PF₆ was added to give a yellow precipitate. The suspension was vacuum filtered and rinsed with water giving a pale-yellow solid (0.1510 g, 86.7% yield) ¹H-NMR (500 MHz, ACN, δ): 8.82 (d, 2H), 8.54 (d, 2H), 5.86 (s, 2H). ¹³C-NMR (126 MHz, CDCl₃, δ): 164.97, 156.44, 148.16, 147.08, 146.04, 145.15, 143.89, 141.92, 139.09, 137.09, 124.91, 117.39, 106.79, 52.03. UV-Vis λₘₐₓ (ACN, M⁻¹cm⁻¹): 401 nm (ε=52000). MALDI-TOF-MS: m/z calculated for C₂₈H₁₁F₁₀N₄S₂ 657.030, found 657.228. Φ_{ACN}=0.65.

Various embodiments of the invention have been described in fulfillment of the various objects of the invention. It should be recognized that these embodiments are merely illustrative of the principles of the present invention. Numerous modifications and adaptations thereof will be readily apparent to those skilled in the art without departing from the spirit and scope of the invention.

1. A thiazolothiazole-based compound of Formula I: 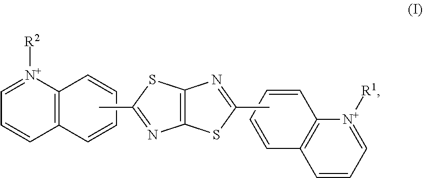
wherein R¹ and R² are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and —OR³, wherein R³ is selected from the group consisting of hydrogen, alkyl, alkenyl, and cycloalkyl.

2. The thiazolothiazole-based compound of claim 1, wherein the thiazolothiazole-based compound is symmetric.

3. The thiazolothiazole-based compound of claim 1, wherein the thiazolothiazole-based compound is asymmetric.

4. The thiazolothiazole-based compound of claim 1 having a difference between the highest occupied molecular orbital (HOMO) and the lowest unoccupied molecular orbital (LUMO) of at least 0.7 eV.

5. The thiazolothiazole-based compound of claim 4, wherein the HOMO/LUMO difference ranges from 2 eV to 4 eV.

6. The thiazolothiazole-based compound of claim 1 having an excited-state reduction potential of at least 2V vs. SCE.

7. The thiazolothiazole-based compound of claim 1 having an excited-state reduction potential of at least 2.2V to 3V vs. SCE.

8. The thiazolothiazole-based compound of claim 1 having an excited-state reduction potential of a reduced species of at least 1V.

9. The thiazolothiazole-based compound of claim 8, wherein the excited-state reduction potential is 1V to 1.6V.

10. A thiazolothiazole-based compound of Formula II: 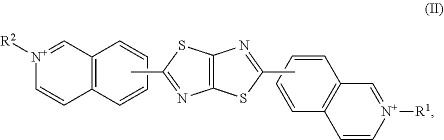
wherein R¹ and R² are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and —OR³, wherein R³ is selected from the group consisting of hydrogen, alkyl, alkenyl, and cycloalkyl.

11. The thiazolothiazole-based compound of claim 10, wherein the thiazolothiazole-based compound is symmetric.

12. The thiazolothiazole-based compound of claim 10, wherein the thiazolothiazole-based compound is asymmetric.

13. The thiazolothiazole-based compound of claim 10 having a difference between the highest occupied molecular orbital (HOMO) and the lowest unoccupied molecular orbital (LUMO) of at least 0.7 eV.

14. The thiazolothiazole-based compound of claim 13, wherein the HOMO/LUMO difference ranges from 2 eV to 4 eV.

15. The thiazolothiazole-based compound of claim 10 having an exited state reduction potential of at least 2V vs. SCE.

16. The thiazolothiazole-based compound of claim 10 having an exited state reduction potential of at least 2.2V to 3V vs. SCE.

17. The thiazolothiazole-based compound of claim 10 having an excited-state reduction potential of a reduced species of at least 1V.

18. The thiazolothiazole-based compound of claim 17, wherein the excited-state reduction potential is 1V to 1.6V.

19. A thiazolothiazole-based compound of Formula III: 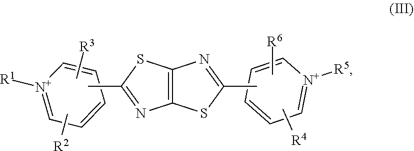
wherein R¹ and R⁵ are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and —OR⁷, wherein R⁷ is selected from the group consisting of hydrogen, alkyl, alkenyl, and cycloalkyl; and
wherein R² and R⁴ are independently selected from the group consisting of alkyl, alkenyl, cycloalkyl, heterocyclyl, halo, aryl, heteroaryl, alkylene-amine, alkylene-aryl, alkylene-heteroaryl, alkylene-heterocyclyl, and —OR⁸, wherein R⁸ is selected from the group consisting of hydrogen, alkyl, alkenyl, and cycloalkyl; and
wherein R³ and R⁶ are independently selected from the group consisting of hydrogen, alkyl, alkenyl, cycloalkyl, heterocyclyl, halo, aryl, heteroaryl, alkylene-amine, alkylene-aryl, alkylene-heteroaryl, alkylene-heterocyclyl, and —OR⁹, wherein R⁹ is selected from the group consisting of hydrogen, alkyl, alkenyl, and cycloalkyl.

20. The thiazolothiazole-based compound of claim 19, wherein the thiazolothiazole-based compound is symmetric.

21. The thiazolothiazole-based compound of claim 19, wherein the thiazolothiazole-based compound is asymmetric.

22. The thiazolothiazole-based compound of claim 19 having a difference between the highest occupied molecular orbital (HOMO) and the lowest unoccupied molecular orbital (LUMO) of at least 0.7 eV.

23. The thiazolothiazole-based compound of claim 22, wherein the HOMO/LUMO difference ranges from 2 eV to 4 eV.

24. The thiazolothiazole-based compound of claim 19 having an excited state reduction potential of at least 2V vs. SCE.

25. The thiazolothiazole-based compound of claim 19 having an excited state reduction potential of at least 2.2V to 3V vs. SCE.

26. The thiazolothiazole-based compound of claim 19 having an excited-state reduction potential of a reduced species of at least 1V.

27. The thiazolothiazole-based compound of claim 26, wherein the excited-state reduction potential of a reduced species is 1V to 1.6V.

28. A thiazolothiazole-based compound of Formula IV: 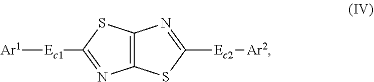
wherein Ar¹ and Ar² are independently selected from the group consisting of 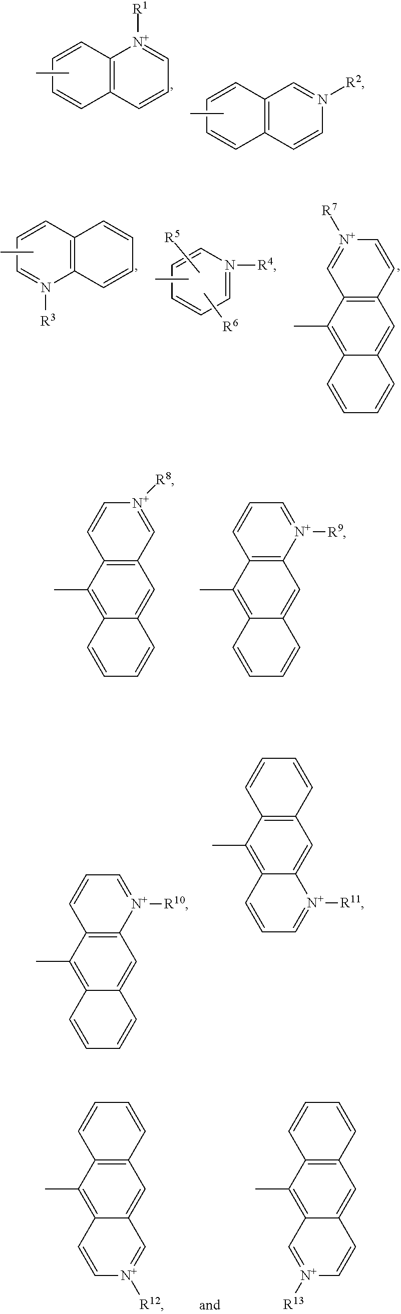
wherein R¹-R⁴ and R⁷-R¹³ are independently selected from the group consisting of alkyl, alkenyl, cycloalkyl, heterocyclyl, aryl, heteroaryl, alkylene-amine, alkylene-aryl, alkylene-heteroaryl, alkylene-heterocyclyl, and —OR¹⁴, wherein R¹⁴ is selected from the group consisting of hydrogen, alkyl, alkenyl, and cycloalkyl; and
wherein R⁵ and R⁶ are independently selected from the group consisting of hydrogen, alkyl, alkenyl, cycloalkyl, heterocyclyl, halo, aryl, heteroaryl, alkylene-amine, alkylene-aryl, alkylene-heteroaryl, alkylene-heterocyclyl, and —OR¹⁵, wherein R¹⁵ is selected from the group consisting of hydrogen, alkyl, alkenyl, and cycloalkyl; and
wherein E_{c1} and E_{c2} are independently selected from the group consisting of a direct bond, arylene, heteroarylene, alkenylene, alkenylene-arylene, alkenylene-heteroarylene, arylene-alkenylene, and heteroarylene-alkenylene.

29. The thiazolothiazole-based compound of claim 28, wherein at least one of E_{c1} and E_{c2} is not a direct bond.

30. The thiazolothiazole-based compound of claim 28, wherein E_{c1} is an aryelene or heteroarylene fused to Ar¹.

31. The thiazolothiazole-based compound of claim 28, wherein E_{c2} is an aryelene or heteroarylene fused to Ar².

32. The thiazolothiazole-based compound of claim 28, wherein the thiazolothiazole-based compound is symmetric.

33. The thiazolothiazole-based compound of claim 28, wherein the thiazolothiazole-based compound is asymmetric.

34. The thiazolothiazole-based compound of claim 28 having a difference between the highest occupied molecular orbital (HOMO) and the lowest unoccupied molecular orbital (LUMO) of at least 0.7 eV.

35. The thiazolothiazole-based compound of claim 34, wherein the HOMO/LUMO difference ranges from 2 eV to 4 eV.

36. The thiazolothiazole-based compound of claim 28 having an excited-state reduction potential of at least 2V vs. SCE.

37. The thiazolothiazole-based compound of claim 28 having an excited-state reduction potential of 2.2V to 3V vs. SCE.

38. The thiazolothiazole-based compound of claim 28 having an excited-state reduction potential of a reduced species of at least 1V.

39. The thiazolothiazole-based compound of claim 38, wherein the excited-state reduction potential is 1V to 1.6V.

40. The thiazolothiazole-based compound of claim 28, wherein at least one of E_{c1} and E_{c2} is a direct bond.

41. The thiazolothiazole-based compound of claim 28, wherein each of E_{c1} and E_{c2} is a direct bond.

42. A method of alkylation comprising:
irradiating a thiazolothiazole-based photocatalyst to place the photocatalyst in an excited state;
forming an alkyl radical via oxidation by the excited state thiazolothiazole-based photocatalyst;
attaching the alkyl radical to an organic substrate to provide an alkylated organic substrate; and
reducing the alkylated organic substrate with the thiazolothiazole-based photocatalyst, wherein the thiazolothiazole-based photocatalyst is selected from the group consisting of the thiazolothiazole-based compound of claim 1, the thiazolothiazole-based compound of claim 10, the thiazolothiazole-based compound of claim 19, and the thiazolothiazole-based compound of claim 28.

43. The method of claim 42, wherein the alkyl radical is formed via oxidation of an organoboron compound.

44. The method of claim 42, wherein the thiazolothiazole-based photocatalyst is placed in the excited state by irradiation with light in the visible region of the electromagnetic spectrum.

45. The method of claim 42, wherein the excited state thiazolothiazole-based photocatalyst has an excited-state reduction potential of at least 2V vs. SCE.

46. The method of claim 45, wherein the excited-state reduction potential is from 2.2V to 3V.

47. The method claim 42, wherein the excited-state thiazolothiazole-based photocatalyst has a reduction potential of at least 1V.

48. The method of claim 47, wherein the redox potential is 1.2 V to 1.6 V.

49. The method of claim 42, wherein the excited-state photocatalyst participates in radical formation via the oxidation.

50. The method of claim 42, wherein the organic substrate comprises an imine.
